(19) United States
(12) Patent Application Publication
(10) Pub. No.: US 2013/0019676 A1
(43) Pub. Date: Jan. 24, 2013

(54) SELF CORRECTING FITNESS TEST

(71) Applicant: Peter T. Durand, Cary, NC (US)

(72) Inventor: Peter T. Durand, Cary, NC (US)

(73) Assignee: RivalHealth LLC

(21) Appl. No.: 13/187,583

(22) Filed: Jul. 21, 2011

(51) Int. Cl. A61B 5/22

(52) U.S. Cl. 73/379.01

(57) ABSTRACT

The present invention relates to a physical fitness test which is easy to do, easy to monitor and is easily self correcting. It involves taking a push, pull, core, and cardio test that is easy to do but can be measured for like age and gender individuals.

### COPYRIGHT NOTICE

A portion of the disclosure of this patent contains material that is subject to copyright protection. The copyright owner has no objection to the reproduction by anyone of the patent document or the patent disclosure as it appears in the Patent and Trademark Office patent files or records, but otherwise reserves all copyright rights whatsoever.

### BACKGROUND OF THE INVENTION

1. Field of the Invention

The present invention relates to a fitness test. In particular, the present invention relates to a fitness test with a self correcting aspect that gives immediate feedback and is difficult to falsify results.

2. Description of Related Art

The measurement of the fitness of an individual is subject to a number of differing assessments. In general, fitness testing is done under the supervision of testing personnel because of the difficulty of performing the test correctly and the tendency for individuals to cheat when left to their own measurements. Measurement of respiratory gases tests a person's maximal oxygen uptake but requires expensive equipment to make an assessment. There are many methods to further asses fitness using sub maximal exercise, but again it is always necessary to have trained personnel observe the testing. Some field tests can be done not requiring exercise equipment but again are difficult and impossible to be utilized without trained personnel.

Other tests can combine measurement of things such as heart rate and speed used performing an exercise, accelerometers and using GPS or other tracking devises. The biggest problem is that without more into these tests are not clear indicators of the improvement of an individual and are difficult or expensive to repeat on a regular basis to measure change in a particular fitness profile.

Currently, there is no system which cost effectively and efficiently measures fitness by an individual at home that can be used on a daily basis or as frequently as desired and where the accuracy of the measurements can be checked by an independent professional to see if the measurements by the individual are/were accurate. Accordingly a more accurate home fitness system is desirable. Compliance is also an issue and the present method allows compliance monitoring as well. Also there is a need to be able to track group fitness as a whole without providing actual data for an individual, i.e. rate a person without data anonymously.

### BRIEF SUMMARY OF THE INVENTION

The present invention relates to a five part fitness test which over comes the problems associated with home fitness testing and can easily be verified after the testing comprising measurement of an individual's weight along with a push, pull, core and cardio test. In addition the measurement, aggregation and presentation of data on a population to identify a baseline and set a target without providing confidential info, is provided.

Accordingly, in one embodiment the present invention relates to a self administered and self reporting fitness test for an individual comprising the method of:
a) the individual performing a maximum number of repetitive push exercises;
b) the individual performing a maximum number of repetitive pull exercises;
c) the individual performing a maximum number of repetitive core exercises;
d) the individual performing a cardio-distance exercise over a specific time;
e) comparing the results of the test to known values for someone of the same age and sex as the individual; and
f) determining the state of fitness of the individual based on the results of the comparison.

### BRIEF DESCRIPTION OF THE DRAWINGS

FIG. 1 is a flow chart of the method of implementation of the present invention.

### DETAILED DESCRIPTION OF THE INVENTION

While this invention is susceptible to embodiment in many different forms, there is shown in the drawings and will herein be described in detail specific embodiments, with the understanding that the present disclosure of such embodiments is to be considered as an example of the principles and not intended to limit the invention to the specific embodiments shown and described. In the description below, like reference numerals are used to describe the same, similar or corresponding parts in the several views of the drawings. This detailed description defines the meaning of the terms used herein and specifically describes embodiments in order for those skilled in the art to practice the invention.

### DEFINITIONS

The terms “a” or “an”, as used herein, are defined as one or as more than one. The term “plurality”, as used herein, is defined as two or as more than two. The term “another”, as used herein, is defined as at least a second or more. The terms “including” and/or “having”, as used herein, are defined as comprising (i.e., open language). The term “coupled”, as used herein, is defined as connected, although not necessarily directly, and not necessarily mechanically.

Reference throughout this document to “one embodiment”, “certain embodiments”, and “an embodiment” or similar terms means that a particular feature, structure, or characteristic described in connection with the embodiment is included in at least one embodiment of the present invention. Thus, the appearances of such phrases or in various places throughout this specification are not necessarily all referring to the same embodiment. Furthermore, the particular features, structures, or characteristics may be combined in any suitable manner in one or more embodiments without limitation.

The term “or” as used herein is to be interpreted as an inclusive or meaning any one or any combination. Therefore, “A, B or C” means any of the following: “A; B; C; A and B; A and C; B and C; A, B and C”. An exception to this definition will occur only when a combination of elements, functions, steps or acts are in some way inherently mutually exclusive.

The drawing featured in the figures is for the purpose of illustrating certain convenient embodiments of the present invention, and are not to be considered as limitation thereto. Term “means” preceding a present participle of an operation indicates a desired function for which there is one or more embodiments, i.e., one or more methods, devices, or apparatuses for achieving the desired function and that one skilled in the art could select from these or their equivalent in view of the disclosure herein and use of the term “means” is not intended to be limiting.

As used herein the term “self administered” refers to a test which is capable of easily and correctly being performed with or without the aid of a physical fitness instructor or some other medical or athletic coach to monitor the progress of the fitness test. In one embodiment, the user reviews a video, e.g. on a website, to teach the user how to conduct the test by themselves. Though one so skilled could later observe the administration of the test to see if the test can actually be performed to the level a person claims to have done, it is clear that it refers to testing capable of being done by an individual without spotting, coaching or the like. Likewise “self reporting” refers to the ability of an individual to not need judgment in measuring the result. In most cases this means either performing a certain exercise a number of times, taking ones weight accurately and measuring the distance one can cover in a limited amount of time. An “individual” refers to a human subject in need of physical fitness testing who can perform and repeat the test over a period of time in order to measure the progress/change of fitness with the test.

The individual that is taking the self administered test first would take their own weight. The weight would best be taken in some repetitive way, i.e. same time of day, same scale, same amount of clothing, and the like but in general the weight of the individual is taken. The test can be done with a less accurate weight as well but the more accurate the weight the better the overall result of the testing. In addition, the individual will need to note their age and their sex for determination of the results of the test in comparison with other individuals of the same or similar sex, weight and age. Such comparison data can either be generated or at least some of the data for individual parts of the test are available in the public domain, for example, the Washington post publishes scores for parts of the test such as sit-ups and push-ups based on age and sex, and such could be modified to include weight by testing multiple individuals. A guide for very poor to superior performance is included. Any standardized data could be utilized or created for the test, e.g. military standards or the like.

The test of the present invention has four different parts to it when taken together form the basis of fitness that has never been put together to monitor the progress of an individual in terms of overall fitness and weight. It has been discovered that weight is directly correlated with the test results, meaning it is difficult if not impossible to cheat on the test because of the discovery that individual weight is a limiting factor in the performance of the entire test. The test consists a repetitive push, pull and core exercise in combination with a cardio-distance exercise for covering a particular distance over a given time. The results of the individual test are then compared to known values of individuals of the same age and sex for determination of fitness relative to that comparison group. Obviously the group can be varied based on general population, a group such as a company or organization, elite athletes, or any comparison group that the individual's fitness is to be measured against. Weight can also be compared, set as a goal, or mentioned as it impacts the results though not the test itself.

As used herein a “repetitive push exercise” refers to an exercise using the arms wherein the triceps and pectorals are the main muscle groups involved in the repetitive exercise. Preference is given to an exercise with little or no equipment. Examples of push exercises include push-ups of all kinds (wide grip, incline, standard, military, etc), weighted triceps extensions, and any triceps based repetitive exercise. The test can be set up to include a maximum number or a maximum number over a given period of time.

As used herein “repetitive pull exercise” refers to an exercise using the arms wherein the biceps and latissimus dorsi are the main muscle groups involved in the repetitive exercise. Preference is given to little or no equipment. Examples of pull exercises are standard pull-ups, chin-ups, wide grip pull-ups, bent over rows, dead lifts, and the like bicep based repetitive exercise. The test can be set up to include a maximum number or a maximum number over a given period of time.

As used herein “repetitive core exercise” refers to an exercise that works the abdominals or other related stomach, chest and mid to lower back muscle groups in a repetitive exercise. Preference is given to little or no equipment. Examples of core exercises are crunches, sit-ups, knee touches, and the like. The test can be set up to include a maximum number or a maximum number over a given period of time.

As used herein a “cardio-distance exercise over a specific time” refers to an aerobic exercise wherein the picking of a particular time for the exercise is done and then allowing the individual to cover the distance by walking, running, or other method and attempting to cover the greatest distance during that time. For example, in one embodiment the time is 12 minutes and the individual covers the greatest distance possible by walking, biking, swimming, elliptical training, running, or the like, or a combination during that time. In general, any time or cardio distance method could be selected but for the purpose of a test it needs to be short enough it can be done easily but long enough to test aerobic fitness. In general, a specific time selected from about 1 minute to about 30 minutes, about 5 to 15 minutes or about 10 to 15 minutes are embodiments of times that could be utilized as a range to select a time for the test. Once a cardio distance test method is selected, it should be utilized in repetitious testing.

Once the test is completed then each of the tests results is compared to the result for a like age and sex individual to determine the fitness compared to other individuals. One of the benefits of this fitness test is that for a given individual progress and results by definition will need to be within certain averages and hence it would be clear or at least suggest based on those parameters when an individual would be guessing or cheating. Under those circumstances the test can be repeated in front of another individual, such as a fitness professional, a coach, a personal trainer, peer, or the like, to test if the test results reported are accurate. By repeating the test at intervals like weekly, monthly, or the like, progress can be checked in a manner that is representative of true health fitness, is easy for the individual to accomplish in a short period of time, and can be checked by repetition in front of a professional where necessary.

Likewise, by tracking the progress of the individual in this manner a goal can be set based on the individual's age and sex and repeated tests can measure the progress toward the goal. The goal can be weight targeted or other desired health metrics.

Now referring to the drawings. The figure represents the method of the test though particular individual exercises could be accomplished in any order. The time for completing the entire test is such that each test is completed within a reasonable time of the next test, a few seconds to minutes between each test though performing each with great times between would also produce results within the scope of the invention that would measure progress. What is important is the test be repeated in the same manner each time.

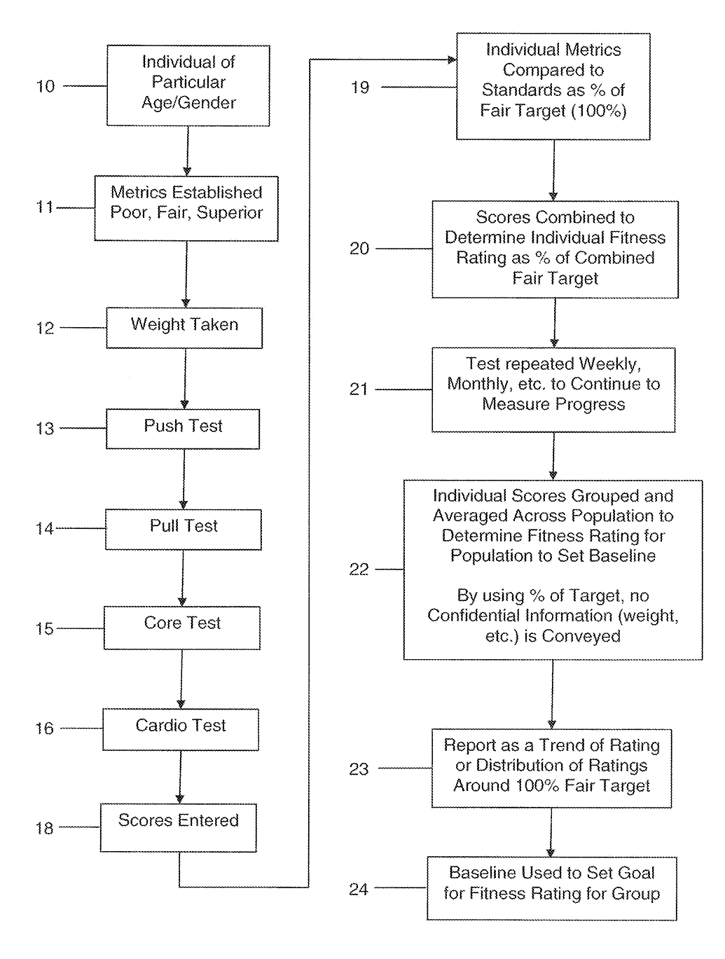

FIG. 1 depicts a flow chart of the implementation of an embodiment of the fitness test of the present invention. An individual of a particular age and sex 10 prepares and is ready to take the fitness test of the present invention. At some point right before, after or during the test, the weight of the individual is optionally taken 12 by normal means scale or the like, metrics are established 11 to get a baseline especially if weight is a goal. The series of tests begin and in any order. For example, the push test 13 is taken doing as many pushups as possible. The pull test 14 is next, accomplished wherein an individual in one embodiment does as many pull-ups as possible. Next a core test 15 is done to test core fitness such as many sit ups as possible by the individual. Lastly, the cardio-distance test 16 is done where an individual traverses the largest distance possible over a selected test time. In one example the cardio test 15 is the greatest distance an individual can cover walking or running in a timed 12 minute period.

Each of the test results 13 through 16 are then individually compared to a standard 16 for that individual of like age and sex to determine where the fitness level is for that individual in comparison to others in a standardized comparison data. The weight 12 can be repeatedly taken to check progress toward weight goals.

Series of the tests are entered 18 into a computer database and then individual metrics are compared to standards as a percent of a fair target 19. Individual metrics are combined to determine and individual fitness rating as a percentage of a combined fair target 20. The test is repeated 21 on a regular basis weekly, monthly, or the like to measure progress.

Scores from individuals are grouped and averaged across a selected population to determine a fitness level compared to those in the select population 22 (e.g. company, city, organization etc.). By utilizing percentage of the target goal rather than actual test results, no confidential info is conveyed to observe group progress. Then a report prepared as a trend of a rating or distribution of rating of the group is conveyed 23 around a target. A baseline can then be utilized to set goals for a fitness for the entire group 24 as a whole without individual data.

The result of the single fitness test gives but one point in time, however, repetition of the fitness test over time gives on a chartable progress of fitness either good or bad such that progress or lack thereof can easily be charted. With the test being simple to implement, hard to cheat on, and not taking much time, this particular fitness test is an improvement over prior tests which did not have the benefits indicated above for a fitness test especially for a self administered fitness test such as the present invention fitness test.

Those skilled in the art to which the present invention pertains may make modifications resulting in other embodiments employing principles of the present invention without departing from its spirit or characteristics, particularly upon considering the foregoing teachings. Accordingly, the described embodiments are to be considered in all respects only as illustrative, and not restrictive, and the scope of the present invention is, therefore, indicated by the appended claims rather than by the foregoing description or drawings. Consequently, while the present invention has been described with reference to particular embodiments, modifications of structure, sequence, materials and the like apparent to those skilled in the art still fall within the scope of the invention as claimed by the applicant.

What is claimed is:

1. A self administered and self reporting fitness test for an individual comprising the method of:
a) the individual performing a maximum number of repetitive push exercises;
b) the individual performing a maximum number of repetitive pull exercises;
c) the individual performing a maximum number of repetitive core exercises;
d) the individual performing a cardio-distance exercise over a specific time;
e) comparing the results of the test to known values for someone of the same age and sex as the individual; and
f) determining the state of fitness of the individual based on the results of the comparison.

2. The fitness test according to claim 1 wherein the push exercise is a push up.

3. The fitness test according to claim 1 wherein the pull exercise is a pull up.

4. The fitness test according to claim 1 wherein the core exercise is a sit up.

5. The fitness test according to claim 1 wherein the cardio exercise is running or walking for the greatest distance the individual can cover over a select period of time.

6. The fitness test according to claim 1 wherein the test is completed repeatedly over a long period of time and repetitions of the test compared to determine the level of progress of fitness of the individual.

7. The fitness test according to claim 1 wherein the test is repeated in front of a fitness professional for determining if the individual is compliant with the test parameters.

8. The fitness test according to claim 1 wherein a fitness goal is set for the individual and the fitness test utilized to determine progress towards the fitness goal.

9. The fitness test according to claim 8 wherein the fitness goal is to achieve a particular weight of the individual.

10. The fitness test according to claim 1 wherein a plurality of individuals in a selected group each perform the test.

11. The fitness test according to claim 10 wherein the group sets a base fitness for the entire group as a whole.

12. The fitness test according to claim 10 wherein the group of individuals repeats the test at intervals to measure group progress as a whole.

13. The fitness test according to claim 10 wherein group sets a target goal.

14. The fitness test according to claim 13 wherein the group test results are reported as a percentage of the target reached.

15. The fitness test according to claim 1 wherein the weight of the individual is taken.
